(19) United States
(12) Patent Application Publication
(10) Pub. No.: US 2008/0248962 A1
(43) Pub. Date: Oct. 9, 2008

(54) Adhesive Bead For Immobilization of Biomolecules and Method For Fabricating a Biochip Using the Same

(75) Inventors: Jae-Kwon Kim, Gyeonggi-do (KR); Eun-Jeong Lee, Jeonnam (KR); Dong Jo Ryu, Daejeon (KR); Jae-Young Jang, Daejeon (KR)

Correspondence Address: MCKENNA LONG & ALDRIDGE LLP, 1900 K STREET, NW, WASHINGTON, DC, 20006, US

(73) Assignee: LG CHEM, LTD., Seoul (KR)

(21) Appl. No.: 11/547,362

(22) PCT Filed: Apr. 24, 2006
(86) PCT No.: PCT/KR2006/001535

§ 371 Date: Jun. 10, 2008

(30) Foreign Application Priority Data
Sep. 15, 2005 (KR) 10-2005-0086284

(51) Int. Cl. C40B 30/04; C12Q 1/00; C40B 50/18; C40B 40/00

(52) U.S. Cl. 506/9; 435/4; 506/32; 506/13

(57) ABSTRACT

The present invention relates to an adhesive bead for immobilizing biomolecules and a method for fabricating a biochip using the same, and more particularly, relates to an adhesive bead functioning both as a solid support immobilizing biomolecules and an adhesive to the surface of a biochip substrate, and a method for fabricating a biochip, the method comprising the steps of immobilizing biomolecules to the adhesive bead to prepare an aqueous suspension of beads on which biomolecules are fixed and fixing the aqueous suspension on a substrate. The adhesive beads of the present invention can be directely immobilized on a biochip without additional equipment and treatment process due to the dual functions of a solid support immobilizing biomolecules and an adhesive to the surface of a substrate.

### TECHNICAL FIELD

The present invention relates to an adhesive bead for immobilizing biomolecules and a method for fabricating a biochip using said adhesive bead, and more particularly, relates to an adhesive bead functioning both as a solid support immobilizing biomolecules and an adhesive to the surface of a substrate, and a method for fabricating a biochip, the method comprising the steps of immobilizing biomolecules to the adhesive bead to prepare an aqueous suspension and fixing the aqueous suspension on a substrate.

2. Background Art

Solid supports capable of immobilizing biomolecules are widely utilized in various biological applications using selective affinity between biomolecules, which include natural supports, such as agarose, cellulose, porous glass, silica, alumina and zeolite and synthetic supports, such as polyacrylamide bead, polymethacrylic acid bead, polystylene bead and membranes (Regnier, F. E., J. Chromatogr. Sci., 14:316, 1976; Hjerten, S., Anal Biochem., 3:109, 1962).

Those solid supports are extensively utilized in the fields of biochips used for high-throughput screening (HTS) and diagnosis as a carrier to fix biomolecules on a biochip substrate in addition to the conventional fields, such as protein purification and/or isolation and affinity chromatography, etc (Sato, K., Adv. Drug Deliv. Rev., 55:379, 2003; Adnerson, H., Electrophoresis, 22:249, 2001; Choi, J. W., Biomed. Microdevices, 3:191, 2001). Solid supports were devised as an alternative way in order to overcome the technical restrictions of two-dimensional fixation, such as self-assembly conventionally used in the related field, namely limitations of integrating biomolecules and maintaining biological activities. The solid supports make it possible for biomolecules to be bio-friendly integrated by confirming biomolecules at high concentration and fixing them on a biochip substrate using the wide three-dimensional surface area of a solid support.

The available solid supports include various types. For example, membrane type utilizes a wide surface area with characteristic pores, as an area for immobilizing biomolecules such as cellulose, and polymer matrix is a support having the fixing area widened and steric hindrance of biomolecules against a substrate improved by forming a thin polymer matrix consisting of bio-friendly polymers, such as glucose, polylysine, chitosan, dextran, polyallylamine and polyvinylalcohol (KR 2004-0004725; Yakovleva, J., Biosens. Bioelectron., 19:21, 2003; Gill, I., Trends in Biotechnology, 18:282, 2000; U.S. Pat. No. 5,034,428;U.S. Pat. No. 5,482,996).

Bead-shaped supports are a fixation support having biomolecules fixed on each spherical bead to collect and thus forming a three-dimensional structure with wide surface area, which can be utilized as a biochip when fixed on a substrate (Sato, K., Adv. Drug Deliv. Rev., 55:379, 2003; Andersoon, H., Electrophoresis, 22:249, 2001; Choi J. W., Biomed. Microdevices, 3:191, 2001). Said membrane and polymer matrices have a couple of drawbacks in that the biomolecule fixation is restricted to the surroundings of the surface in contact with the outside, or it is difficult to maintain biological activities of enzymes and other proteins sensitive to outside environment when the biomolecules are covalently bound for high fixation rate. On the contrary, bead supports have great advantages in that they have high utilization rate of the surface area due to three-dimensional structure formed using each bead on which biomolecules are immobilized and various immobilization methods maintaining biomolecule activity can be utilized. Especially, bead supports can serve as an appropriate material to facilitate biochip fabrication in the manufacturing process of biochips that needs to immobilize biomolecules within microchannels, such as lab-on-a-chip due to its easy handling.

In addition, the traditional beads have a disadvantage in that they require alternative ways to fix beads in microchannels since they don t have adhesiveness to substrates. The conventional ways to fix beads are a method of confining beads within microchannels using physical partitions, a fixation method using magnetic fields and a method using ultrasound or Laser tweezer. However, those methods have disadvantages in that they have a limitation on selecting beads and complicate fabrication process for biochips, causing noise during photomeasurement, and requiring auxiliary equipment inside or outside of a biochip. Thus they are not cost-effective to be applied for lab-on-a-chip (Sato, K., Adv. Drug Deliv. Rev., 55:379, 2003; Andersoon, H., Electrophoresis, 22:249, 2001; Choi, J. W., Biomed. Microdevices, 3:191, 2001; Meng, A., Transducers, Sendai, Japan, 876, 1999; Dorre, K., Bioimaging, 5:139, 1997).

Meanwhile, the inventors have filed an application (KR 10-2004-104944) disclosing a method for fabricating a biochip, the method comprises immobilizing probes or beads having probes fixed, on the surface of a substrate using an adhesive. According to said patent, it is possible to fix beads having probes fixed on a substrate by an adhesive without using physical partitions or magnetic fields, but this still needs a supplement of an additional adhesive.

Therefore, there have been desperate needs for the development of an adhesive bead which functions as a support fixing biomolecules and an adhesive to the surface of a biochip substrate to be fixed directly on a biochip without additional equipment and treatment, and a biochip using the same.

### SUMMARY OF THE INVENTION

Accordingly, the present inventors have made extensive efforts to develop an adhesive bead functioning both as a solid support fixing biomolecules and as an adhesive adhering to the surface of a biochip substrate to be immobilized on a biochip without additional equipment and treatment, and a biochip using the adhesive bead, thereby completing the present invention.

The main object of the present invention is to provide an adhesive bead functioning both as a solid support fixing biomolecules and as an adhesive adhering to the surface of a biochip substrate, and a method for preparing the same.

Another object of the present invention is to provide a method for fabricating a biochip, the method comprises attaching biomolecules to the adhesive bead to prepare an aqueous suspension of the bead on which biomolecules are fixed; and then immobilizing the aquous suspension on a substrate, and a biochip fabricated by the method.

In order to achieve the above object, the present invention provides an adhesive bead functioning both as a solid support fixing biomolecules and as an adhesive adhering to the surface of a chip substrate, which is prepared by emulsifying a hydrophilic monomer, a mainmonomer and a comonomer in an aqueous medium; and polymerizing the aquous suspension.

In the present invention, the hydrophilic monomer is preferably one or more selected from the group consisting of methacrylic acid, acrylic acid, itaconic acid, hydroxyethylmethacrylate, hydroxypropylmethacrylate, acrylamide, glycidyl methacrylate, polyethyleneglycol acrylate, polyethyleneglycol methacrylate, palitoleic acid, oleic acid, lenoleic acid, arachidonic acid, linolenic acid, allylalcohol and vinylalcohol. The mainmonomer is preferably one or more selected from the group consisting of butadiene, ethylacrylate, butylacrylate, ethylhexylacrylate and octylacrylate. The comonomer is preferably one or more selected from the group consisting of vinyl acetate, acrylonitrile, acrylamide, styrene, methylmethacrylate, and methylacrylate.

The present invention also provides a method for preparing an adhesive bead, the method comprises: (a) obtaining an emulsion by adding monomer(s) to a aqueous solution of an emulsifier; (b) stirring the mixture of said emulsion obtained in the step (a) and a solution which is prepared with hydrophilic monomer(s) in an aqueous medium and then heated up to about 75° C. in N₂ ambient; and (c) carrying out polymerization by adding a polymerizing initiator to the emulsion obtained in the step (b).

In the method for preparing the inventive adhesive bead, said aqueous medium is preferably one or more selected from the group consisting of water, ethanol, methanol, DMF, DMSO, acetone and NMP. The hydrophilic monomer is preferably one or more selected from the group consisting of methacrylic acid, acrylic acid, itaconic acid, hydroxyethylmethacrylate, hydroxypropylmethacrylate, acrylamide, glycidyl methacrylate, polyethyleneglycol acrylate, polyethyleneglycol methacrylate, palitoleic acid, oleic acid, lenoleic acid, arachidonic acid, linolenic acid, allylalcohol and vinylalcohol.

In the method for preparing the inventive adhesive bead, said monomer preferably contains at least one mainmonomer selected from the group consisting of butadiene, ethylacrylate, butylacrylate, ethylhexylacrylate and octylacrylate, and at least one comonomer selected from the group consisting of vinyl acetate, acrylonitrile, acrylamide, styrene, methylmethacrylate and methylacrylate. The combination ratio of the mainmonomer to the comonomer is preferably determined by glass transition temperature (Tg) of the adhesive bead, wherein the Tg is preferably 0-45° C. lower than the temperature when biochips are fabricated or used.

In the method for preparing the inventive adhesive bead, said emulsifier is preferably one or more selected from the group consisting of sodium lauryl sulfate, gelatin, methylcellulose, polyvinylalcohol, cetyltrimethyl-ammonium bromide and sodium olelate. Said polymerizing initiator is preferably at least one selected from the group consisting of potassium persulfate, ammonium persulfate, azo-bis-isobutyronitrile (AIBN) and bezoyl peroxide (BPO).

The present invention also provides a method for fabricating a biochip, the method comprises: (a) preparing an aqueous suspension of adhesive beads to which biomolecules are fixed, by attaching biomolecules to the adhesive beads; (b) attaching said aqueous suspension on a chip substrate.

In the method for fabricating the inventive biochip, the step (b) preferably comprises: spotting the aqueous suspension on the substrate; and attaching the adhesive bead on the substrate by drying. Said spotting is preferably performed by inkjetting. Said method for attaching biomolecules to the adhesive bead is preferably performed by any one method selected from the group consisting of hydrophobic absorption, covalent binding and electrostatic attraction.

In the method for fabricating the inventive biochip, said biomolecule is any one selected from the group consisting of nucleic acids, amino acids, proteins, peptides, lipids, carbohydrates, ligands, cofactors and enzyme substrates. Said chip substrate is preferably any one selected from the group consisting of a microwell, a slide substrate and a microchannel of lab-on-a-chip. The material of said chip substrate is preferably one or more selected from the group consisting of polymethylmethacrylate, polycarbonate, polystyrene, cyclic olefin copolymers, polynorbornene, styrene-butadiene copolymers, acrylonitrile butadiene styrene, glass, silicon, hydrogels, metals, ceramics and porous membranes.

The present invention also provides a biochip produced by the method described above, and having adhesive beads to which biomolecules are attached, fixed on a substrate.

The present invention also provides a method for detecting a target substance in a sample, the method comprises: (a) applying a sample containing a target substance to the biochip; and (b) detecting a target substance specifically bound to the biomolecule on said biochip.

Detection of the target substance in a sample is preferably performed by one or more methods selected from the group consisting of a detecting method using biolabels, such as radioactive isotopes, luminescent or colorizing dyes, enzyme-linked immunoassay (ELISA) using bioenzyme, electrochemical immunoassay, particle turbidimetric immunoassay, and a detecting method using fluorophore.

The present invention also provides a biochip for determining infection of HBV (Hepatitis B virus) resistant to Lamibudin; immobilized with beads on the chip substrate, and said beads are the adhesive beads, any one claim among claims 1 to 4, attached with SNPs (single nucleotide polymorphism) of SEQ ID NO: 1 or 2.

The present invention also provides a concavo-convex structure partially or entirely coated with the adhesive bead on the surface of a biochip substrate.

Other features and examples of the invention are clearly described in more detail in the following description and claims.

### BRIEF DESCRIPTION OF THE DRAWINGS

FIG. 1 is a scanning electron microscope image of the inventive adhesive beads (600 nm) immobilized on a substrate, magnified 33 10,000.

FIG. 2 is a scanning electron microscope image of the beads in FIG. 1, magnified ×900,000.

FIG. 3 is a graph showing size variations of beads depending on the amount of the injected emulsifier.

FIG. 4 is a graph showing Tg variations of beads depending on copolymerization ratio of mainmonomers and comonomers.

FIG. 5 is scanning electron microscope images of beads having the glass transition temperatures (Tg) different from each other.

FIG. 6 is a graph showing that surface coverage of biomolecules on beads is increased as the concentration of biomolecules goes high.

FIG. 7 is a graph showing that surface coverage of biomolecules on polystyrene beads and the inventive adhesive beads is increased with the reaction time for fixing biomolecules on beads.

FIG. 8 is scanning electron microscope images of an aqueous suspension containing adhesive beads spotted on a polymethylmethacrylate substrate according to the percentage by weight of each.

FIG. 9 is a scanning electron microscope image of a concavo-convex structure fabricated by dip coating with an aqueous suspension of adhesive beads on a plastic substrate.

FIG. 10 is a graph obtained by spotting an aqueous suspension of the adhesive beads having a protein fixed, on a biochip substrate and measuring auto fluorescence of the formed spots to quantify.

FIG. 11 is a graph showing that the non-specific binding was quantified by spotting an aqueous suspension of the adhesive beads having a protein fixed, on a biochip substrate and treating with a non-specific protein on the formed spots.

FIG. 12 is a photograph of fluorescence scanning in which S-adenosyl-L-homocysteine (SAH) are detected at various concentrations by competitive immunoassay using the inventive biochip.

FIG. 13 is a graph showing the result of performing competitive immunoassay for the target substance, SAH using the inventive biochip and Maxisorp of Nunc Co.

FIG. 14 is a photograph of fluorescence scanning in which SNPs (single nucleotide polymorphism) of oligonucleotides having the sequence of HBV polymerase gene are detected at various concentrations using the inventive biochip.

FIG. 15 is a graph showing fluorescence signal intensity by analyzing the photograph of FIG. 14.

FIG. 16 is a graph showing a photograph of fluorescence scanning in which a protein antigen, Immunoglobulin G (IgG) is detected using the inventive biochip by direct immunoassay.

FIG. 17 is a graph showing fluorescence signal intensity by analyzing the photograph of FIG. 16.

### DETAILED DESCRIPTION OF THE INVENTION AND PREFERRED EMBODIMENTS THEREOF

The present invention relates to an adhesive bead functioning both as a solid support immobilizing biomolecules and as an adhesive to the surface of a substrate, a method for producing the same, a biochip having a bead in which biomolecules are attached to the adhesive bead, immobilized on a substrate, and a method for fabricating the same. Each step of the method for fabricating the inventive biochip is described as follows.

Step 1: Preparation of an Aqueous Suspension Containing Adhesive Beads

The inventive adhesive bead refers to a solid material with adhesive property in an aqueous suspension and comprises a mainmonomer conferring adhesiveness, a comonomer conferring rigidity, and a hydrophilic monomer for water dispersion.

The inventive adhesive bead can be prepared by mixing a mainmonomer, a comonomer and a hydrophilic monomer in an aqueous medium and polymerizing using the conventional methods, for example, suspension, emulsion, dispersion, microemulsion, miniemulsion, reverse emulsion and the like. The condition of polymerization determines various diameters of beads to be produced. For using as a fixative support of biomolecules, generally beads need to have diameters ranging from a few dozen nanometers to a few microns.

In addition, two functions of said bead as an adhesive and a support can be conferred by manipulating the combination ratio of a mainmonomer and a comonomer comprising beads, particularly, by selecting the combining ratio of copolymerization which enables both characteristics to be expressed simultaneously in the condition of biochip application, using the adhesive characteristic of mainmonomers with flexibility and stickiness, and the characteristic of comonomers with rigid solidity. The important factor determining the combination ratio of a mainmonomer and a comonomer is the intrinsic glass transition temperature (Tg) of prepared beads, said Tg is preferably 0˜45° C. lower than the temperature when a biochip is fabricated or used. For instance, if a biochip is fabricated or used at room temperature (25° C.), Tg of an adhesive bead preferably ranges −15˜25° C. that is 0˜45° C. lower than room temperature, and more preferably −15˜10° C.

Step 2: Preparation of an Aqueous Suspension Containing Adhesive Beads with Biomolecules Fixed on

The inventive biochip utilizes an adhesive bead having wide surface area as a medium in order to increase the immobilization density of biomolecules to be fixed on a biochip substrate. Immobilization methods of biomolecules on beads include hydrophobic adsorption that directly triggers the immobilization with the hydrophobic surface of a bead itself, covalent bonding that uses a particular reaction group of copolymer chain comprising beads, and electrostatic attraction. The aqueous medium used to prepare an aqueous suspension of said bead may include any solvent with an aqueous characteristic. That is, the aqueous medium can be water, ethanol, methanol, DMF, DMSO, acetone and NMP. However, it is not limited thereto. Preferably, water can be used.

Step 3: Spotting of an Aqueous Suspension of Beads

The suspension of beads having biomolecules immobilized can be fixed on the surface of a biochip substrate by spotting the suspension on the substrate. For said spotting, any spotting method traditionally used in the art may be used, generally spotting by inkjet printing may be used. It is advantageous to use inkjet printing since it facilitates quantitative spraying of the inventive aqueous suspension of beads on a substrate.

Various substrates used in the field of biochips may be used as a substrate for fixing adhesive beads, representatively a microwell, a slide substrate, a microchannel of lab-on-a-chip can be used, but it is not limited thereto. Also, materials for the substrates may be selected from the group consisting of polymethylmethacrylate (PMMA), polycarbonate (PC), polystyrene (PS), cyclic olefin copolymers, polynorbornene, styrene-butadiene copolymers (SBC), arylonitrile butadiene styrene, glass, hydrogels, silicon, metals, ceramics and porous membranes, but it is not limited thereto.

Step 4: Drying

Traditional drying methods used for fabricating a biochip by spotting can be used, for example, drying at room temperature. The optional temperature is determined according to materials, such as 15˜33° C. for proteins and 15˜90° C. for DNA.

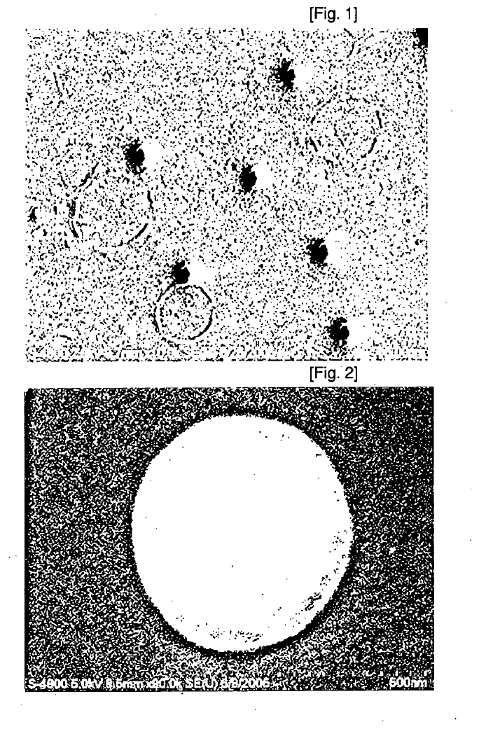

As a result of scanning electron microscopy of a biochipfabricated by the process described above, as shown in FIG. 1 and FIG. 2, it was confirmed that the beads were bound to the substrate by the surface interaction between the beads and the substrate as the drying progresses. Therefore, a biochip can be fabricated using the adhesive bead according to the present invention.

Application of Biochip Produced by the Present Invention

The present invention can be applied to quantitative analysis and presence test of target molecules existing in a sample and comprises the steps of fixing adhesive beads having biomolecules immobilized, on a biochip substrate by spotting, applying a sample containing a target molecule to be detected and detecting a target molecule specifically bonded with said biomolecule.

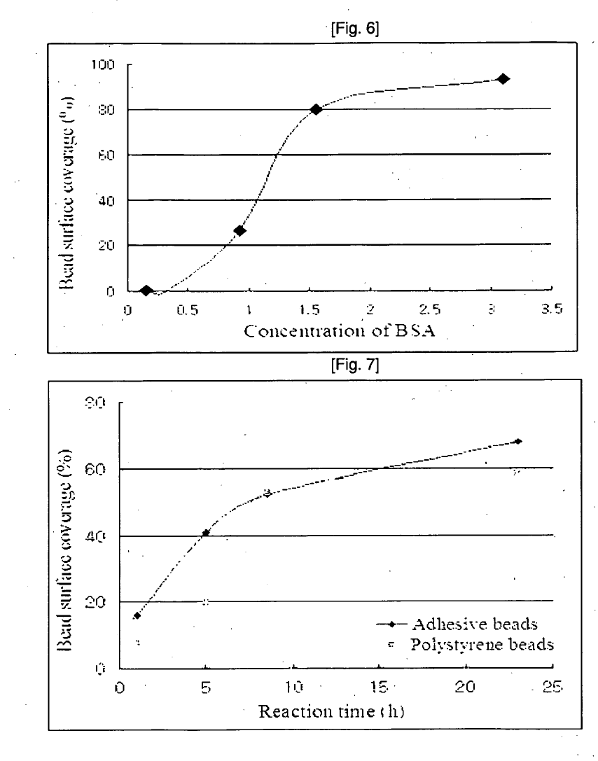

In addition, the inventive adhesive bead may be used for the fabrication of a concavo-convex structure consisting of beads by coating the beads themselves entirely or partially on the surface of a substrate (FIG. 7). The dimensional structure has many useful functions in a biochip, for example, it is applied to a detection part to be used as wide-surfaced substrates for directly spotting biomolecules to fix, or applied to specific microchannels of lab-on-a-chip using capillary flow to be used as a fluid delaying part by hydrophobic flow retardation.

### EXAMPLES

Hereinafter, the present invention will be described in more detail by examples. It is to be understood, however, that these examples are for illustrative purpose only and are not construed to limit the scope of the present invention.

### Example 1

### Preparation of an Aqueous Suspension Containing Adhesive Beads

A main reactor was added with 622.1 g of deionized water and 3.5 g of itaconic acid to heat up to about 75° C. in N₂ atmosphere. Another reactor was added with an emulsion obtained by mixing 35.0 g of butylacrylate, 31.4 g of methylmethacrylate, 0.1 g of allylmethacrylate, 1.2 g of an aqueous solution of 3% by weight sodium lauryl sulfate.

When the temperature of the main reactor was stabilized, the emulsion prepared in said another reactor was transferred to the main reactor and stirred more than 1 hour for enough emulsification. And then 7.0 g and 1.0 g of an aqueous solution of 3% by weight potassium persulfate was added, respectively to allow the reaction for 2 hours, thus obtaining polymers containing adhesive beads.

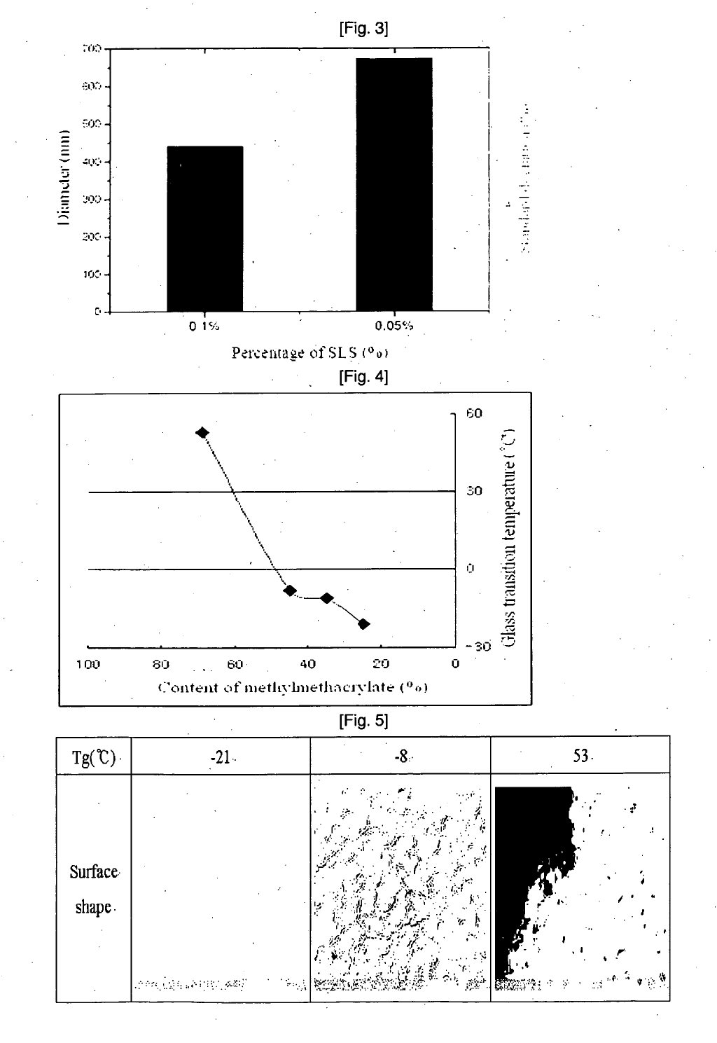

Said polymers were washed by dialysis and an ion-exchange resin, and diluted in deionized water to prepare an aqueous suspension containing adhesive beads. The diameters of said adhesive beads can be regulated by the amount of an emulsifier. FIG. 3 shows the result of analyzing diameters of beads produced at various amounts of an emulsifier, sodium lauryl sulfate, which revealed that submicro-sized beads on average were produced when 0.1˜0.05% by weight emulsifier was added.

Tg of an adhesive bead, a copolymer, generally corresponds to a median value of those of polybutylacrylate as a mainmonomer and polymethylmethacrylate as a comonomer. Therefore, an adhesive bead with desired Tg could be produced by regulating combination ratio of each monomer (FIG. 4).

FIG. 5 is scanning electron microscope image of beads having Tg different from each other, which showed that beads with low Tg had strong adhesiveness to easily stick on a substrate but failed to have dimensional structures due to film formation, whereas beads with high Tg had strong solidity to maintain a distinct bead-shape but failed to stick on a substrate due to its fragility when dried at room temperature. Therefore, beads with Tg ranging between −15˜10° C. is proper when dried at room temperature.

### Example 2

### Immobilization Efficiency According to the Concentration of Biomolecules

Since the surface of beads prepared in Example 1 has both an area for adhering to a substrate and an area for immobilizing biomolecules, the application efficiency of adhesive beads which is the object of the present invention can be regulated by controlling the area occupied by biomolecules of the whole surface area of a bead.

An aqueous phosphate buffer (pH 7.2) solution of bovine serum albumin as biomolecules to be fixed were prepared at concentrations of 0.16, 0.31, 0.93, 1.55 and 3.10 mg/ml and mixed with an aqueous suspension containing 2% by weight said adhesive beads at 1:1 ratio, followed by shaking for 14 hours at room temperature for reaction. After terminating the reaction, the supernatant was collected by centrifugation to quantify for the amount of bovine serum albumin fixed on beads by measuring the remaining amount of bovine serum albumin without being fixed (FIG. 6). As a result, as shown in FIG. 6, the coverage rate of a bead surface could be regulated by controlling the reaction amount of biomolecules to be fixed.

### Comparative Example 1

### Measurement of Immobilization Rate According to Reaction Time and Comparative Test with Polystyrene Beads

The amount of biomolecules immobilized on the bead was measured with reaction time, and compared with a commercially available polystyrene beads. 1.6 mg/mL of an aqueous phosphate buffer (pH 7.2) solution of bovine serum albumin and an aqueous suspension containing 2% by weight adhesive beads (diameter 510 nm) prepared in Example 1 and polystyrene beads (diameter 600 nm) were prepared to be subjected to immobilization process as in Example 2. The surface coverage was measured with the reaction time as biomolecules are immobilized on beads (FIG. 7). As a result, as shown in FIG. 7, it was confirmed that surface coverage of biomolecules could be regulated by controlling reaction time in the case of the adhesive bead prepared in Example 1 and they showed as excellent immobilization capacity as commercially available polystyrene beads.

### Example 3

### Shape of Spots According to the Concentration of Adhesive Beads

The shape of spots formed on a substrate is affected by the concentration of beads in dispersed bead aqueous suspension in fabricating a biochip using adhesive beads. 0.05, 0.1, 0.5, 1% by weight bead aqueous suspensions containing the adhesive beads (average diameter 510 nm, Tg −8° C.) prepared in Example 1 were prepared, respectively, and each 0.5 mL of the aqueous suspension was spotted on polymethyl-methacrylate substrates. Then, the substrates were dried for 12 hours at room temperature and the surface shape of each spot was observed (FIG. 8). As a result, as shown in FIG. 8, it is revealed that the density of attached adhesive beads increases as the concentration of beads in a spot augments, and the multilayered fixation of adhesive beads, if the concentration of beads exceeds 0.5% by weight, advances film formation due to the behavior of a polymer chain.

### Example 4

### Formation of a Concavo-Convex Structure by Coating Adhesive Beads

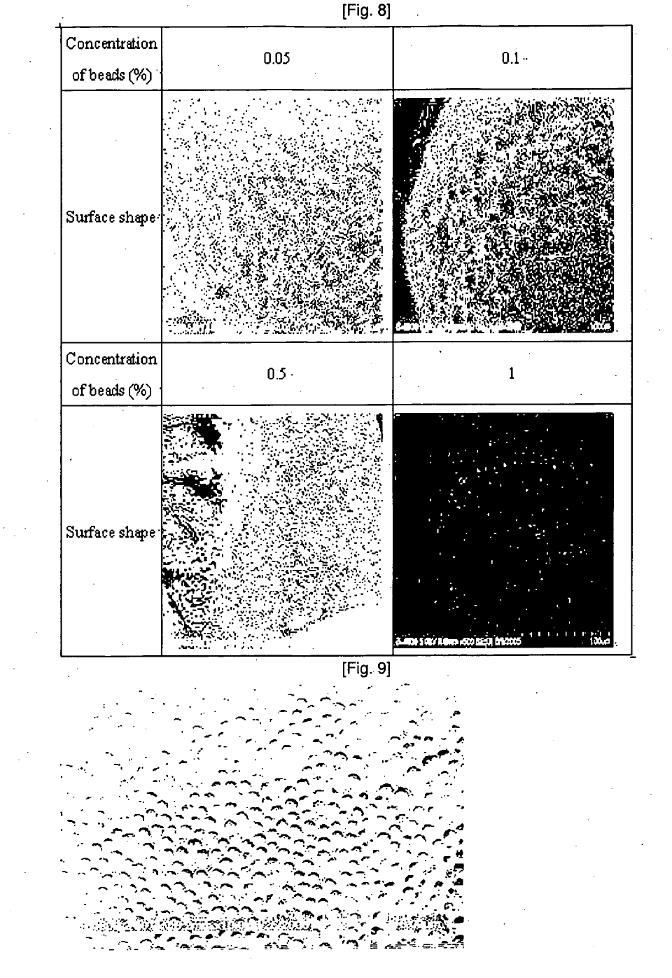

The bead aqueous suspension containing 8.5% by weight adhesive beads produced in Example 1 (diameter 510 nm, Tg −8° C.) was subjected to dip coating partially or entirely on the surface of a polymethylmethancrylate substrate (FIG. 9). As a result, as shown in FIG. 9, it was confirmed that a concavo-convex structure comprised of single-layered beads was formed. This concavo-convex structure can be applied to a fluid delaying part or wide-surfaced substrates in biochips.

### Example 5

### Autofluorescence Measurement and Non-Specific Binding of Proteins of Spots

As a preliminary test for applying the inventive adhesive beads to biochips, a biochip substrate was nano-spotted by an aqueous suspension of beads and measured for the intensity of autofluorescence and non-specific binding.

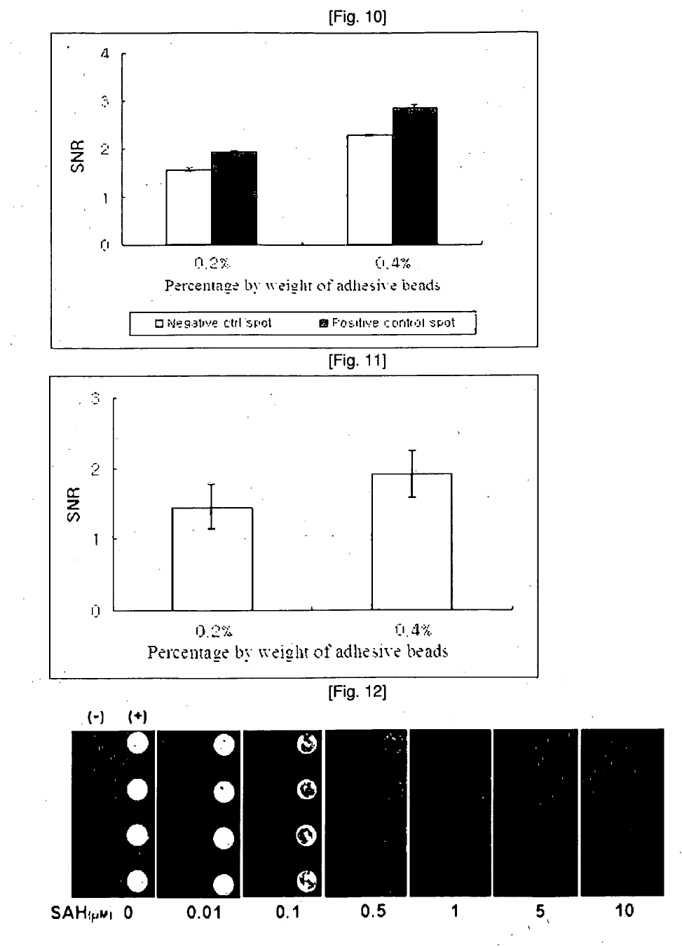

200 [symbol] of 3.1 mg/ml of bovine serum albumin (BSA) or an aqueous phosphate solution of BSA labelled with SAH(S-adenosyl-L-homocysteine) was mixed with 200 [symbol] of an aqueous suspension containing 2% by weight adhesive beads produced in Example 1 to shake for 15 hours at room temperature for reaction. After the reaction, it was contrifugated and washed, followed by preparing an aqueous suspension containing 0.2 and 0.4% by weight adhesive beads. On a polymethylmethacrylate (PMMA) substrate, said aqueous suspension containing adhesive beads was spotted at the volume of 50 nL using an inkjet arrayer and the substrate was measured for autofluorescence of the fixed spots using fluorescence image scanner (Axxon) (FIG. 10). As a result, as shown in FIG. 10, spot autofluorescence of adhesive beads was less than 3 signal to noise ratio (SNR).

Meanwhile, adhesive beads coated with BSA that were spotted by the same way described above were treated with an aqueous solution of anti-SAH antibody and quantified for non-specific binding of proteins (FIG. 11). The result shown in FIG. 11 confirmed that fluorescence intensity of spots measured after nonspecific binding reaction of anti-SAH antibody was less than 3 signal to noise ratio(SNR), showing that non-specific binding of proteins ignorably occurred.

The result that autofluorescence and non-specific binding is not significant indicates that said beads did not interfere with detecting the fluorescence of target molecules during the reaction between the inventive adhesive beads and target molecules. In conclusion, this fact showed that bead supports of the present invention can be adequately used for biochips.

### Example 6

### Competitive Immunoassay on a SAH Target Substance

In order to fabricate a biochip capable of detecting a target substance, SAH, using the same method as in Example 5, BSA and SAH-labelled BSA were coated, respectively on adhesive beads and prepared an aqueous suspension of 0.4% by weight beads using phosphate buffer as an aqueous medium. A PMMA substrate was spotted with the prepared bead aqueous suspension at the volume of 50 nL, dried for 30 minutes at 30° C. and for 20 hours at room temperature, blocked with phosphate buffer (pH 7.4) containing 3% by weight BSA and 0.05% by volume Tween for 30 minutes, and washed. For fluorescence detection, Cy3-labelled secondary antibody and anti-SAH antibody were pre-incubated for 30 minutes and mixed with SAH at various concentrations as a target substance to be detected, and then subjected to a competitive immune reaction with the spots on the biochip.

Photographs of fluorescence scanning and detection results of spots according to the concentrations of SAH were shown in FIG. 12 and FIG. 13 (-?-), respectively. Fluorescence signal was represented as a relative value based on the signal value set to 100 when a target substance, SAH was not added. In the detection of SAH by competitive immunoassay of the biochip produced in the present example, fluorescence signal decreased by more than 90% compared with the case without a target substance, SAH, and the highest detection limit was about 2˜5 μM.

### Comparative Example 2

### Comparison with Two-Dimensional Fixation of Biomolecules

In order to prove superiority of three-dimensional fixation by the adhesive beads of the present invention, the efficiency of immunoassay detection was compared with that of the conventional two-dimensional fixation which is commercially available as biomolecule fixation.

MaxiSorp chip of Nunc Co. as a typical biochip capable of two-dimensional fixation of biomolecules was selected and examined for competitive immunoassay on SAH target substance to compare the result with that of Example 6.

Each of 0.5 mg/ml of BSA and SAH(S-adenosyl-L-homocysteine)-labelled BSA were respectively added in phosphate buffer containing 20% by volume glycerol and spotted on MaxiSorp chip to dry for 20 hours in a humidity chamber. After washing the spotted chips, competitive immunoassay was performed as in Example 6. The result shown in FIG. 13(-▪-) showed that maximum decreased value of fluorescence signal by competitive reaction was about 50% and standard quantitation ranged from 0.01 to 0.5 μM, indicating much less detection level than that of fixation method using adhesive beads in Example 6.

The results described above were due to drawbacks of the conventional two-dimensional fixation using MaxiSorp chip, such as low intergration efficiency of biomolecules and steric hindrance to the biochip surface. Therefore, the biochip using the adhesive beads of the present invention proved relatively superior to conventional chips.

### Example 7

### Detection of Specific SNPs

Single nucleotide polymorphism (SNP) which is one of DNA detection applications was detected by immobilizing oligonucleotide sequences used for examining infection of type B hepatitis virus (HBV), having resistance to a therapeutic agent, Lamibudin on the adhesive bead.

HBV resistant to Lamibudin is a virus in which YMDD motif of virus polymerase is mutated. YIDD mutant having isoleucine substituted for Met 552 is typical. There is only one base difference between normal sequence expressing YMDD motif and a mutant sequence expressing YIDD motif.

TABLE 1
Probes and Target Sequences for SNP detection

| | |
|---|---|
| | SEQ |
| | ID |
| Sequence | NO: |

| | | | |
|---|---|---|---|
| Probe | Nomal | 5′-NHhd 2-TC AGT TAT AT G GAT GAT GTG-3′ | 1 |
| | Mutant | 5′-NH₂-TC AGT TAT ATC GAT GAT GTG-3′ | 2 |
| Target* | | 5′-Cy3-CAC ATG ATC CAT ATA ACT GA-3′ | 3 |

*Oligonucleotide composing HBV polymerase gene sequence

Each of a normal probe(SEQ ID NO:1) complementary to the sequence of HBV polymerase gene having YMDD motif and a mutant probe(SEQ ID NO:2) complementary to the gene sequence of YIDD mutant were immobilized on a biochip surface using the adhesive beads to examine for selective detection of fluorescence-labelled HBV polymeras gene sequence (a target) (Table 1).

In order to immobilize each oligonucleotide probe efficiently on adhesive beads, each oligonucleotide probe was coupled with BSA using sulfosuccinimidyl 4-(N-maleimidomethyl)cyclohexane-1-carboylate and coated on adhesive beads using similar method to Example 6.

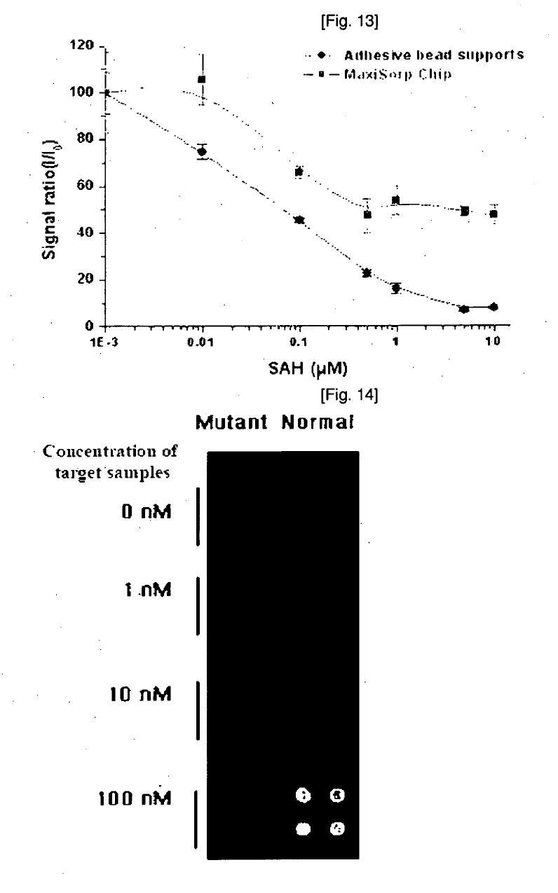

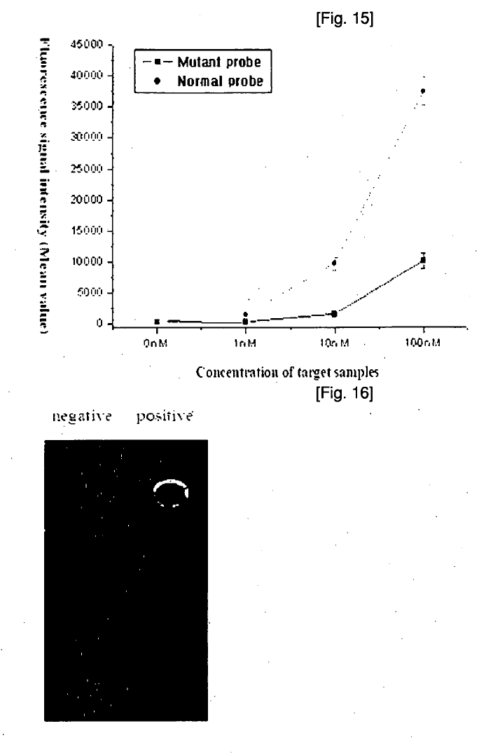

DNA chip was fabricated by nanospotting 50 nL of 0.4% by weight bead aqueous suspension on a plastic substrate. The biochip was washed with deionized water for 3 minutes, treated with 80 [symbol] of blocking solution (3 ml of 20×SSC, 1.35 ml of formamide, 500 [symbol] of 1% by weight BSA, 150 [symbol] of deionized water) for 30 minutes at 40° C., and added with an additional 5 [symbol] of target sample (0 nM˜100 nM), followed by hybridizing for 1 hour at 40° C. To eliminate target sequences binding non-specifically, the biochip was washed for 10 min with 2×SSC and for another 10 minutes with 0.2×SSC to analyze by fluorescence scanner. FIG. 14 is a photograph showing fluorescence scanning of SNPs of oligonucleotides having DNA sequence of HBV polymerase, detected at various concentrations using the biochip of the present example, and FIG. 15 is a graph showing fluorescence signal intensity by analyzing the photograph of FIG. 14.

The analysis of Fluorescence signal intensity from the scanning photograph showed that the normal probe complementary to the target sample (target molecules) had 4.1-fold higher discrimination ability compared with the mutant probe having one base difference. Therefore, the biochip of the present invention proved to be useful for DNA detection, such as SNP.

### Example 8

### Direct Immunoassay on Protein Target Substance

Whether immunoassay on protein antibodies besides a target substance having low molecular weight, such as SAH in Example 6 using the adhesive beads is possible, was examined. BSA (Bovine serum albumin) was used as a protein antigen. Using similar method to Example 6, a simple biochip to detect immunoglobulinG (IgG) was fabricated by preparing adhesive beads coated with BSA (Calbiochem Co., antigen grade) and spotting the beads on a PMMA substrate.

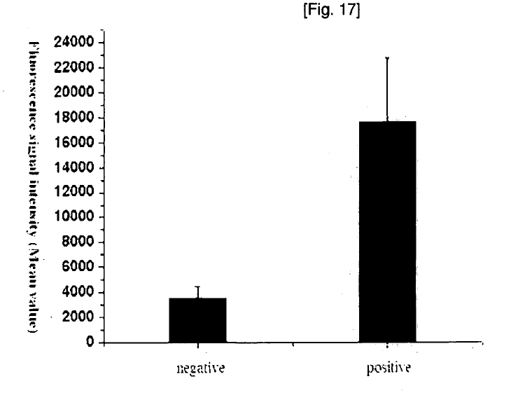

For blocking the surface, 30% human serum in an aqueous solution of 1× PBS buffer was treated on the chip for 30 minutes at room temperature anti-SAH IgG (a polyclonal antibody, 50 [symbol] /ml) as a negative control and anti-BSA IgG (a monoclonal antibody, 50 [symbol] /ml) as a positive control were selected and allowed to react on said biochip substrate for 45 minutes at room temperature, and then anti-mouse-Cy3 (polyclonal, 10 [symbol] /ml) labelled with Cy3 fluorescence dye as a secondary antibody were allowed to react for 20 minutes at room temperature. After remaining antibodies binding non-specifically were washed with an aqueous solution of 1× PBS buffer, fluorescence signal was detected using the fluorescence scanner. FIG. 16 is a photograph showing fluorescence scanning of a target substance, Immunoglobilin G (IgG), detected by direct immunoassay using the biochip of the present invention, and FIG. 17 is a graph showing fluorescence signal intensity by analyzing the photograph of FIG. 16.

The analysis of fluorescence signal intensity from the scanning photograph showed that the positive control specific to a target substance, IgG had 7.7 (positive signal intensity/negative signal intensity) of detection ability, compared with the negative control nonspecific to IgG. Therefore, the biochip of the present invention proved to be useful for the detection of protein antibodies.

### INDUSTRIAL APPLICABILITY

The adhesive beads of the present invention can be directely immobilized on a biochip without additional equipment and treatment process due to the dual functions of a solid support immobilizing biomolecules and an adhesive to the surface of a substrate. Also, the adhesive beads of the present invention has an advantage in that the surface coverage of the beads can be regulated by the reaction amount of biomolecules and reaction time for immobilizing biomolecules on the beads. Furthermore, the adhesive beads have high immobilization density of biomolecules compared with conventional two-dimensional fixation of biomolecules while showing similar immobilization capacity to that of commercially available existing beads and thus making it possible to fabricate biochips in a small size. Therefore, it is highly cost effective.

Although the present invention has been described in detail with reference to the specific features, it will be apparent to those skilled in the art that this description is only for a preferred embodiment and dose not limit the scope of the present invention. Thus, the substantial scope of the present invention will be defined by the appended claims and equivalents thereof.

### Sequence Listing

1. DNA(Artificial Sequence)

probe: tcagttatat ggatgatgtg

2. DNA(Artificial Sequence)

probe: tcagttatat cgatgatgtg

3. DNA(Artificial Sequence)

probe: cacatgatcc atataactga

1. An adhesive bead functioning both as a solid support immobilizing biomolecules and as an adhesive adhering to the surface of a chip substrate, which is prepared by a method comprises: emulsifying a hydrophilic monomer, a mainmonomer and a comonomer in an aqueous medium; and polymerizing the aqueous emulsion.

2. The adhesive bead according to claim 1, wherein the hydrophilic monomer is one or more selected from the group consisting of methacrylic acid, acrylic acid, itaconic acid, hydroxyethylmethacrylate, hydroxypropylmethacrylate, acrylamide, glycidyl methacrylate, polyethyleneglycol acrylate, polyethyleneglycol methacrylate, palitoleic acid, oleic acid, lenoleic acid, arachidonic acid, linolenic acid, allylalcohol and vinylalcohol.

3. The adhesive bead according to claim 1, wherein the mainmonomer is one or more selected from the group consisting of butadiene, ethylacrylate, butylacrylate, ethylhexylacrylate and octylacrylate.

4. The adhesive bead according to claim 1, wherein the comatiomer is one or more selected from the group consisting of vinyl acetate, acrylonitrile, acrylamide, styrene, methylmethacrylate, and methyacrylate.

5. A method for preparing an adhesive bead, the method comprises:
(a) obtaining an emulsion by adding monomer(s) to an aqueous solution of an emulsifying agent;
(b) stirring the mixture of said emulsion obtained in the step (a) and a solution which is prepared with hydrophilic monomer(s) in an aqueous medium and then heated up to about 75° C. in N2 atmosphere; and
(c) carrying out polymerization by adding a polymerizing initiator to the emulsion obtained in the step (b).

6. The method for preparing an adhesive bead according to claim 5, wherein said aqueous medium is one or more selected from the group consisting of water, ethanol, methanol, DMF, DMSO, acetone and NMP.

7. The method for preparing an adhesive bead according to claim 5, wherein said hydrophilic monomer is one or more selected from the group consisting of methacrylic acid, acrylic acid, itaconic acid, hydroxyethylmethacrylate, hydroxypropylmethacrylate, acrylamide, glycidyl methacrylate, polyethyleneglycol acrylate, polyethyleneglycol methacrylate, palitoleic acid, oleic acid, lenoleic acid, arachidonic acid, linolenic acid, allylalcohol and vinylalcohol.

8. The method for preparing an adhesive bead according to claim 5, wherein said monomer is one or more selected from the group consisting of butadiene, ethylacrylate, butylacrylate, ethylhexylacrylate and octylacrylate; and said co-monomer is preferably one or more selected from the group consisting of vinyl acetate, acrylonitrile, acrylamide, styrene, methylmethacrylate and methyacrylate.

9. The method for preparing an adhesive bead according to claim 5, wherein said emulsifier is one or more selected from the group consisting of sodium lauryl sulfate, gelatin, methylcellulose, polyvinylalcohol, cetyltrimethyl-ammonium bromide and sodium olelate.

10. The method for preparing an adhesive bead according to claim 5, wherein said polymerizing initiator is one or more selected from the group consisting of potassium persulfate, ammonium persulfate, azo-bis-isobutyronitrile (AIBN) and bezoyl peroxide (BPO).

11. The method for preparing an adhesive bead according to claim 8, wherein the combination ratio of the mainmonomer to the comonomer is preferably determined by glass transition temperature (Tg) of the adhesive bead, wherein Tg is 0-45° C. lower than the temperature when biochips are fabricated or used.

12. A method for fabricating a biochip, the method comprises:
(a) preparing an aqueous suspension of the adhesive bead on which biomolecules are fixed by attaching biomolecules to the adhesive bead of claim 1; and
(b) attaching said aqueous suspension on a biochip substrate.

13. The method for fabricating a biochip according to claim 12, wherein the step (b) comprises: spotting the aqueous suspension on the substrate; and attaching the adhesive bead on the substrate by drying.

14. The method for fabricating a biochip according to claim 12, wherein said spotting is performed by inkjetting.

15. The method for fabricating a biochip according to claim 12, wherein said method for attaching biomolecules to the adhesive bead is performed by any one method selected from the group consisting of hydrophobic adsorption, covalent bonding and electrostatic attraction.

16. The method for fabricating a biochip according to claim 12, wherein said biomolecule is any one selected from the group consisting of nucleic acids, amino acids, proteins, peptides, lipids, carbohydrates, ligands, cofactors and enzyme substrates.

17. The method for fabricating a biochip according to claim 12, wherein said substrate is any one selected from the group consisting of a microwell, a slide substrate and a microchannel of lab-on-a-chip.

18. The method for fabricating a biochip according to claim 17, wherein the material of said substrate is any one selected from the group consisting of polymethylmethacrylate, polycarbonate, polystyrene, cyclic olefin copolymers, polynorbornene, styrene-butadiene copolymers, acrylonitrile butadiene styrene, glass, silicon, hydrogels, metals, ceramics and porous membranes.

19. A biochip fabricated by the method of claim 1 and having an adhesive bead on which biomolecules are attached, fired on a substrate.

20. A method for detecting a target substance in a sample, the method comprises:
(a) applying a sample containing a target substance to the biochip of claim 19; and
(b) detecting a target substance specifically bound to the biomolecule on said biochip.

21. The method for detecting a target substance according to claim 20, wherein said detection of the target substance in a sample is performed by one or more methods selected from the group consisting of a detecting method using biolabels, such as radioactive isotopes, luminescent or colorizing dyes, enzyme-linked immunoassay (ELISA) using bioenzyme, electrochemical immunoassay, particle tubidimetric immunoassay, and a detecting method using fluorophore.

22. A biochip for determining infection of HBV (Hepatitis B virus) resistant to Lamibudin; immobilized with beads on the chip substrate, and said beads are the adhesive beads, claim 1, attached with SNPs (single nucleotide polymorphism) of SEQ ID NO: 1 or 2.

23. A concavo-convex structure partially or entirely coated with the adhesive bead of claim 1 on the surface of a substrate.


SEQUENCE LISTING (3 sequences)

SEQ ID NO: 1 - DNA, 20 bp, Artificial Sequence
  oligonucleotide probe for Hepatitis B virus
tcagttatatggatgatgtg

SEQ ID NO: 2 - DNA, 20 bp, Artificial Sequence
  oligonucleotide probe for Hepatitis B virus
tcagttatatcgatgatgtg

SEQ ID NO: 3 - DNA, 20 bp, Artificial Sequence
  Oligonucleotide from Hepatis B virus polymerase gene
cacatgatccatataactga